(19) United States
(12) Patent Application Publication
(10) Pub. No.: US 2020/0093418 A1
(43) Pub. Date: Mar. 26, 2020

(54) System and method for optimized monitoring of joints in physiotherapy

(71) Applicants: Andrei Alexandru Kluger, Bucuresti (RO); Camil Dragos Moldoveanu, Bucuresti (RO)

(72) Inventors: Andrei Alexandru Kluger, Bucuresti (RO); Camil Dragos Moldoveanu, Bucuresti (RO)

(73) Assignee: Kineto Tech Rehab SRL, Bucharest (RO)

(21) Appl. No.: 16/269,576

(22) Filed: Feb. 7, 2019

(30) Foreign Application Priority Data
Sep. 21, 2018 (RO) A/00710

(51) Int. Cl. A61B 5/00; A63B 24/00

(52) U.S. Cl. CPC A61B 5/4528; A63B 24/0003; A63B 2220/40; A61B 5/6801; A61B 5/0002

(57) ABSTRACT

The invention relates to a set of motion sensing devices HS mounted above and below the injured joint, a mobile application MobApp that receives sensor data, and a web server WS. The mobile application comprises a physiotherapists interface MPTI and a patient interface MPI, an Exercise definition module EDM, an assessment module ASMD and the movement analysis module MAM. The physiotherapist interface MPTI shows the patients their progress, the list of scheduled exercises, receives feedback and delivers assessments. The web server WS hosts a cloud-based database DB which keeps the relevant data, a Machine learning unit MLU and a physiotherapist interface WPTI. The claimed method, ensures an optimized monitoring of the physiotherapy of joints, by neutralizing within the Calibration and compensation unit CCU the influence of the position and drift of the motion sensors and also by implementing the Machine learning unit MLU and relevant algorithm.

### TECHNICAL FIELD

The invention presents a new system and the corresponding implementing method of monitoring patients in need of physiotherapy, both in the clinic and at home, by using three axes accelerometers and three axes gyroscopes as wireless motion sensors, attached around the injured joint, and detecting, monitoring and reporting the quality of the exercises patients are doing both to the physiotherapist and to the patient

It is based on a mobile application that receives sensor data and a web server where exercises and exercise programs are defined and scheduled for patients, by hosting a cloud-based database which keeps the physiotherapist, patient and exercise data.

Present invention is an optimized and customer oriented monitoring solution, by allowing both flexible placement of the motion sensors above and below the patient's affected joint, minimisation of the linear drift of the gyroscopes as well as an optimization of the exercise program based on big data analysis, derived from a gradual improvement of the program quality, learned from the patient's adherence and the previous responses of the physiotherapist

### BACKGROUND ART

Background art reveals an important number of solutions built up around mobile applications and dedicated to systems and related methods for monitoring the process of rehabilitation by physiotherapy of the injured joints.

Practically all of them are using one or more devices for motion and/or position detection of the patient during the physiotherapy recovery process of the joints, a database containing relevant information on exercises, modules for comparing real execution with model exercise and making relevant assessments and means for particular calibration of the input information from sensors.

The solution as presented in (1), relates to a system and implementing method that proposes monitoring and correction of the execution of physical exercises by means of a Pilates type exercises device. Detection is ensured by a fixed Kinect motion sensor plus a video camera together with an infrared proximity sensor.

Accordingly, this solution is based on image processing and the calibration is intended not to detect position of the sensors around the joint but to adjust the position of the body and the exercises to the position of the sensors on the injured joint, which might create real and sensitive calibrating problems.

In (2), the described monitoring system and method is based on inertial sensors and a data processor for determining the movements of the patient and their correction against a predefined model. The implemented calibration procedure has the meaning of calibration using start and end positions of a specific movement and assumes these positions are fixed and known. If those positions are not achieved with accuracy by the patient, which in practice happens often, the errors of the calibration will be reflected in the subsequent angle measurement and will lead to incorrect measurements.

In document (3), the described monitoring system and method is practically based on an unspecified number and types of motion and position sensors: accelerometer, gyroscope, magnetometer, MEMS sensor, digital compass, inertial meter, temperature sensor, video camera, etc. Accordingly, the processor for sensor data is specific to the actually used sensors. An important limitation of this solution would be the lack of a specific module/algorithm for position calibration of the sensors.

Likewise, in (4), the number and types of sensors in the Sensors unit are not effectively specified, by leaving to the user the option of correlating with the software in the mobile application. The proposed calibration has the exclusive meaning of a selection of the rehabilitation programs and does not cover the usual calibration of the sensors' position.

In document (5), the entire solution of the monitoring system and related method, including the calibration unit is based on processing video information. Possible disadvantages of that solution reside in the impossibility of detecting the supine position, detection only for privileged angles (where the camera is not obstructed by the body) and diminished accuracy.

Patent document (6) describes a system where sensors are mounted on the patient's limbs in order to monitor the exercises done by the patient. The system uses sensors with a three axis accelerometer, three axis gyroscopes and three axis compass.

Using a compass in a clinic environment means the sensor data is subjected to magnetic interferences from all the equipment available in the surroundings of the patient and data can be influenced and distorted.

The solution presented in said document requires an accurate calibration of the data from the sensors, which implies that one should know exactly the position of the sensors placed on the limb in order to measure accurately the angles between the segments of the limb.

Some limitations appear to result from the cited background art, which indicates needs for improved or, more specific, optimised solutions.

The first one relates to sensitivity and accuracy of the data processed by the motion and position sensors used by respective systems, usually improved by calibration means.

Virtually all known systems, are based on a calibration procedure which assumes a very well determined position of the sensors around the injured joint, a process which could be inherently inaccurate, sometimes difficult and subject to local condition which could undermine the calibration process. If used by itself, the gyroscope has a linear drift which could contribute to an alteration of the information. The same is true for the accelerometers that have a high static noise. The compass is also very prone to magnetic errors when close to iron objects.

Besides problems derived from the cited patent literature, an overview of the background art reveals that progress on the flexion is now reported either by subjective means (depending on the physiotherapist's experience and knowledge) or by using a goniometer. The goniometer isn't very accurate and cannot be used all the time during the exercise. For an injured patient it's critical to see the evolution of the rehabilitation process, what he is doing wrong while performing the exercises and this information is now lacking.

Most of solutions are using only a limited 2D representation and rendering of the injured joint and having the relevant angles displayed on a 3D representation on the mobile interface would allow the patient to have an objective information about his/her momentary progress and how he can strive to improve.

Recovery programs are not standardized; each physiotherapist is free to choose his own exercise programs, which means patients will not have the same quality of service across clinics or even the same clinic. There's only a very small amount of studies to determine which exercises work best for each type of affection. There are exercises that are prescribed for each type of affection but there's no additional data correlating the adherence and progress to the exercises and type of patient.

A very important progress indicator is the adherence of the patient to the treatment prescribed by the physiotherapist and this too isn't measured accurately, most of the time being subjectively checked by the physiotherapist or self reported by the patient (when doing the exercises at home).

There's great standardization in the surgeries performed by the doctors, but almost none in the recovery process, either recovery in the clinic or recovery at home. When measuring the success of the patient's recovery most of it is made from the physiotherapy which follows the surgery and only a small part depends on how well the surgery was done.

### SUMMARY OF INVENTION

The technical problem raised and solved by this invention is minimizing and neutralizing the disturbing effect of the position of the sensors around the injured joint under physiotherapy monitoring, compensation of the gyroscope's dynamic drift and, based on the analysis of the received data, optimising the quality and timing of the prescribed exercises and thus ensuring the success in the patient's recovery.

The solution of the present invention is built up within a mobile application and comprises a set of motion sensing devices mounted above and below the injured joint of a patient, a mobile application that receives sensor data and offers interfaces to the physiotherapist and the patient, relevant exercise definition, assessments and movement analysis, and respectively a web server which hosts a cloud-based database which keeps the physiotherapist, patient and exercise data. It allows the physiotherapist to prescribe a set of exercises, accurately monitor the patient's training and to objectively measure the progress of the patient in the clinic and at home which provides much more accurate info than the subjective one that might be offered by the patient.

Some important technical features have been considered by this invention in order to contribute in achieving an optimized physiotherapy monitoring of joints.

Firstly, the claimed invention calibrates the signals received from the motion sensors by a specific calibration algorithm which detects the actual positioning and ensures the orientations of the sensors is related to the actual physical axis of rotation of the joint, and thus the patient should not fix them in a certain position. This makes the solution very simple to use both for the physio in assessments and for the patients when performing the remote assisted exercises at home.

Secondly, the claimed invention ensures a dynamic gyro drift compensation which ensures the sensor data is immune to drift.

Another technical feature of the claimed invention is the implementation of a machine learning procedure meant to ensure a continuous assessment of the exercise program changes and program evaluation. The progress of the patient's flexion is automatically analyzed against patterns already existing in the database and depending on the detected improvements; it generates relevant notifications, alerts, and, finally, a better selection and scheduling of the exercises and automatic personalization of the patient schedule.

Having regard to similar solutions contained in the state of the art, one may conclude that this invention offers the following advantages:
motion sensors are limited to accelerometer and gyroscope;
by using calibration and compensation procedures, the solution is “flexible” to the placement of the said sensors anywhere above and below the affected joint, and compensates the dynamic drift of the gyroscope;
solution ensures determination of the actual physical flexion axis of the joint with respect to the anatomical characteristics of the patient;
solution enables detection and measurement of additional angles: abduction/adduction angle, hip flexion angle and hip rotation angle
enables an automatic optimization of the exercise detection by using a machine learning procedure and thus, improves the patient's adherence by accurately identifying the “improved” movement the patient should do in order to recover more rapidly

### BRIEF DESCRIPTION OF DRAWINGS

A detailed description of the invention and its embodiments will be further presented with reference to the following figures:

FIG. 1—General system structure

FIG. 2—Calibration and correction procedure

FIG. 3—Movement analysis procedure

FIG. 4—Exercise definition procedure

FIG. 5 Patient exercise assistance procedures

FIG. 6 Machine learning procedure

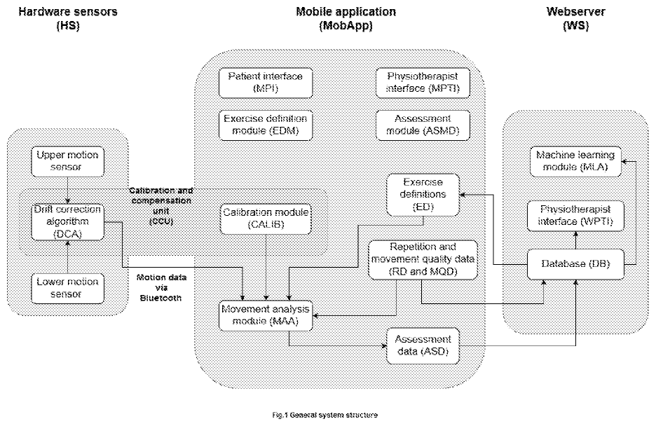

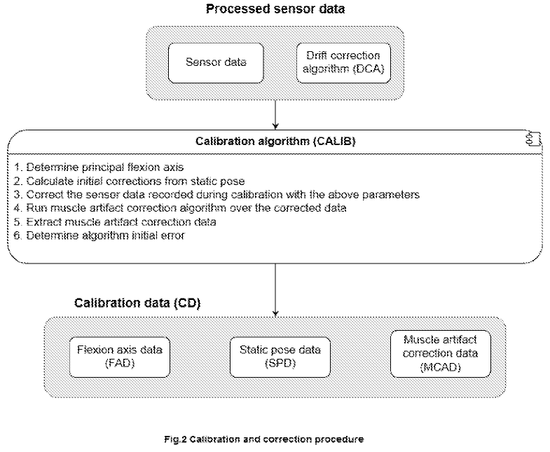

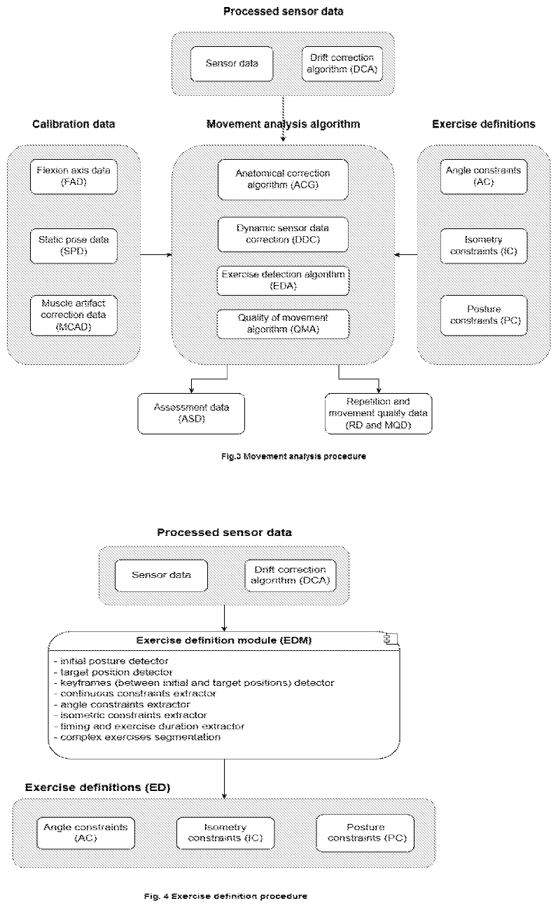

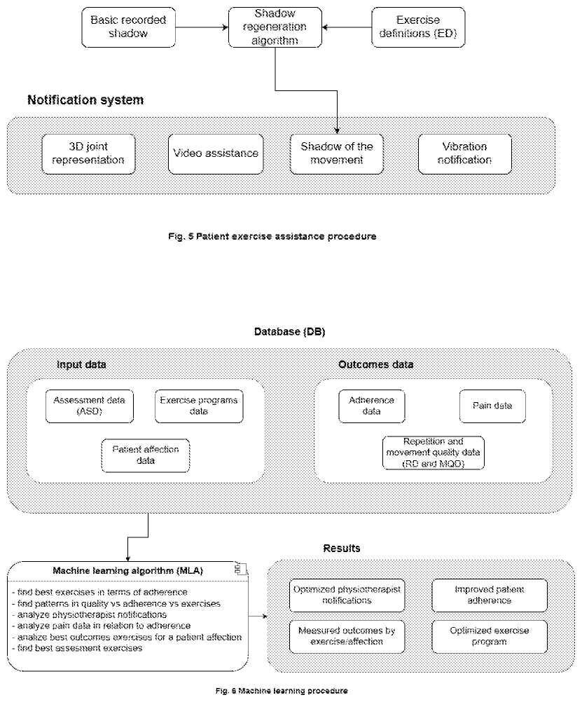

### DESCRIPTION OF EMBODIMENTS

The solution consists of a set of motion sensing devices HS mounted above and below the injured joint of a patient, a mobile (tablet or mobile device) application MobApp that receives sensor data, a web server WS where exercises and exercise programs are defined and scheduled for patients, a cloud-based database DB which keeps the physiotherapist, patient and exercise data.

The hardware sensors HS contain three axis accelerometers, three axis gyroscopes. Different from other motion tracking implementations, the system does not use a magnetometer as it's very susceptible to interference with iron based objects, which are very common in the physiotherapy clinics and even at home.

Data from the accelerometer and gyroscope is fused within the device to obtain a corrected position in space. This data offers the orientation in the absolute reference system of the sensor without it having a true north heading as the solution doesn't use the compass. By itself, the data offered by the each sensor is not relevant and it has to be processed by the mobile application in relation to the joint and the other sensor.

Using only gyroscopes and accelerometers without compass means that the orientation is prone to drift on the long term. A compensation procedure formalized within a related algorithm DCA compensates linear drift from the gyroscopes.

The placement of the sensors on the body is very important for other solutions as it influences all the subsequent measurements. This is why other solutions rely on very careful placement of the sensors that can be recognized by their algorithms. From our perspective it's very hard for the patient to accurately place the sensors in the desired position: the desired position might be over the injury, it's not comfortable to wear the sensors in those positions, the patient has different sized limbs than the standard, etc. Another issue is that careful placement means lost time and additional worries for the patient who should concentrate on recovering from the injury. This might affect the adherence to the treatment plan which is not desirable.

This is why the claimed solution allows the motion sensing devices to be placed randomly above and below the patient's affected joint. The patient doesn't have to fix them in a certain position; the calibration algorithm detects the actual positioning and ensures that the orientations of the sensors are related to the actual physical axis of rotation of the join.

The mobile application MobApp is made of two separate interfaces, a physiotherapists interface MPTI and a patient interface MPI. These two different interfaces are accessible using the corresponding login parameters, if the user logging in is a patient the patient interface is shown, if the user is a physiotherapist, the physiotherapist interface is shown instead.

The mobile application MobApp includes as well an Exercise definition module EDM, an Assessment module ASMD and a Movement analysis algorithm MAA.

The physiotherapist interface MPTI shows all the patients that physiotherapist has registered, their progress, the list of scheduled exercises, feedback from the patient and patient assessment module.

The patient assessment module ASMD allows the physiotherapist to objectively measure the phase the patient is in the recovery process, how well the injured joint behaves compared to the healthy one and, based on this, change the thresholds of exercises or even the exercise list, and compared also with an average individual, in the same age and activity group.

This assessment, and the data associated with it—ASD, is an extremely important part of the recovery process as in this phase the physiotherapist determines with the patient what is the expected outcome of the physiotherapy. This outcome can be functional recovery, professional athlete performance or anywhere in between. This outcome determines the length of exercise program, the intensity of the training, the intermediate goals and the actual exercises to be done.

By using the initial assessment and monitoring the patient all through the recovery process the patient is more motivated and has more feedback about how the recovery is progressing which leads to greater adherence to the program.

The physiotherapist has a database of exercises to choose from or predefined programs that can assign to a patient depending on the affection/surgery.

The exercise database contains a list of predefined exercises that the physiotherapist will use as a base for his own exercise programs. Each exercise can be customized within the physiotherapist application so it fits the patient using the system.

An exercise definition module allows the physiotherapist to record an exercise with the sensors on his body (or alternatively to instruct the patient to do an exercise with the sensors on) and then the exercise processing algorithm disseminates the data and sets all the constraints based on that recording. This allows the physiotherapist on one hand to easily customize the existing exercises and on the other hand enables him to create his own set of exercises if the ones in the platform are not enough.

The patient interface PMI contains a list of exercises prescribed by the physiotherapist to the patient using the web application. This list can be updated by the physiotherapist using the web platform or the mobile application. The mobile application receives data from the motion sensing devices, shows the movement live on the mobile device screen, counts exercise repetitions and shows how correct the movement was done using the thresholds the physiotherapist has previously set.

A calibration and compensation procedure formalized by the calibration algorithm CALIB and drift correction algorithm DCA is used on one side to ensure motion data is aligned with physical axis of movement and on the other side that the sensor data are drift immune. The algorithm uses a combination of a static pose and dynamic movements of the patients' joint to determine where the motion detection devices were placed on the patient's body and uses that information to correct the movement data accordingly.

The dynamic calibration movement is made around the main flexion axis of that joint i.e. for knee the motion is a knee flexion, for elbow it's an elbow flexion motion, for back it's a back bending motion, etc. This motion, repeated a number of times gives the raw principal axis of that joint FAD. Sensor positioning is inferred from this principal axis and then body position compensation is calculated. This uses previously determined muscle models, compensating for the movement of the sensors on the patient's body due to the muscle tissue and thus obtaining the muscle artefact correction data—MCAD.

A static pose, and the data that comes with it—SPD, is also taken during the calibration process to align the absolute coordinate systems of the two sensors. This static pose assumes that the joint of the patient should be straight and not flexed. If the patient cannot keep the joint straight as in the first days after surgery, the procedure allows for a correction angle inputted manually by the physiotherapist which is used to offset all the subsequent data from the sensors. This ensures the motion data is correct even for these specific cases where the patient is unable to fully extend the joint.

Further compensation is made taking into consideration the anatomical characteristics of the joint around which the sensors are placed. If the joint has certain degrees of freedom, the motion is checked and corrected against those parameters. For example, if it's a hinge joint there shouldn't be rotational movements in the recorded calibration motion.

Starting from the assumption that the gyroscope drift is linear with time, the double derivative of the angles returned by the gyroscope is calculated in the DCA algorithm, thus removing the linear part of the drift. To be able to reconstruct the angle a double integration is used which adds two sets of constants (one set for each integration operation). The first set is determined from the samples where the joint is not moving (in the static pose phase), for those samples it's equal to zero. The second one is determined as the initial orientation, and could be found in (7).

The drift is further eliminated when the two sensors readings are synchronized using the acceleration from the two sensors. This exploits the fact that the acceleration of one segment of the joint is the sum of the acceleration of the joint's center and the acceleration of that segment around the center. We apply this knowledge to determine the joint's center position in the local coordinates of each sensor allowing the angles to be determined with much reduced drift.

In order to implement the movement analysis procedure via the related movement analysis algorithm MAA, the relevant exercises are defined as a sequence of constraints that have to be met and an initial position that must be matched for the exercise to be started. The constraints are categorized by level of importance into layers, with layer 1 being the most important and layer 3 the least important. Each layer can have multiple constraints depending on the complexity or requirements of the exercise. Constraints can be defined as the target value, the tolerance of that value, when to check the respective value, if it's an isometric constraint and how much the patient needs to keep the body segments in that isometric position.

The time when the constraints are checked can be: on the peak of the movement, in the initial position, in the final position or continuous where the rule must apply to the whole exercise time.

The algorithm defines some standard initial postures from where the exercise starts, like standing, sitting, supine, side-lying, etc. but allows custom postures too. These custom postures are defined either manually by adding posture constraints PC or automatically where the physiotherapist uses the device to record a new exercise, by using the Exercise definition module—EDM and the algorithm analyses the collected data and generates an exercise definition ED complete with all the associated constraints AC, IC and PC.

Data from the motion detection devices is processed and fed through an Anatomical correction procedure and related algorithm, ACG, and a Dynamic sensor data correction module DDC. The ACG detect if any anatomical defined constraints were breached and assesses the breach and decides if the movement should be corrected accordingly. The DDC applies all the calibration data to the processed sensor data coming from the hardware sensors HS.

Both ACG and DDC are used to correct sensor placement adjustment, muscle movement and sensor decalibration during the time exercises are performed, and ensure the sensors keep their alignment to the meaningful physical axis at all times.

The movement data is then compared to the current exercise definition in an Exercise detection procedure and related algorithm EDA. If the exercise was done within the limits defined by the physiotherapist, a grade is generated in a Quality of movement algorithm QMA that represents the correctness of the exercise, taking into account the difficulty of the exercise and how well the constraints imposed by the physiotherapist were achieved. Visual, text and audio notification are sent to the patient to inform of the correctness of the exercise allowing the patient to improve the quality of the repetition.

An Exercise definition module EDM allows the physiotherapist to create a new exercise by putting sensors on the affected joint and doing the exercise he wants to add. The EDM extracts the definition of the exercise, in terms of constraints and lets the physiotherapist change it quickly.

The EDM records calibrated movement data, i.e. data that was corrected for sensor positioning on the body, and for muscle artefacts from the hardware sensors HS and then analyses that data in order to create the new exercise.

Initially, the initial posture recorded is compared to the standard postures already in the database. If there are major differences, a new custom posture is defined using posture constraints. These are expressed in angles and rotations that the application can measure. For example if it's a knee joint there can be hip angle, flexion angle, abduction angle, etc. If the sensors are placed on the back there can be lower back rotation, flexion, side inclination, upper back inclination, etc. The angles can be calculated from one segment to the other or one segment to the body.

The target position is defined similar as the initial posture, using the angles that the application can measure.

If the exercise is a complex one and can be expressed as a succession of linear movement it is segmented into several parts and for each part a target and initial position is defined. This works similar to graphic animation where, from a sequence of key frames and a linear interpolation between them, an animation can be constructed.

Some of the angle constraints must be met during the whole exercise (for example keep the back straight while flexing it; keep the elbow straight while raising a weight with your hand or the knee straight while raising the foot in front of you). These are continuous constraints and the EDM suggests this too to the physiotherapist.

Some exercises count on keeping the joint in a certain position for an amount of time—these are called isometric exercises and are an important part of the recovery process. EDM also allows detection of isometric exercises and changing the time that should be spent in isometry.

EDM also analyses the data recording and extracts a timing for the exercises as some of them need to be done in a certain amount of time (either they need to be faster than an interval or they need to be slower than an interval).

An Assessment module ASD allows the physiotherapist to evaluate the patient at the start/end of important recovery phases and decide if the patient can go to the next phase or even finish the recovery process.

An assessment is made of a set of exercises specifically tailored to check the progress of the patient in certain areas that are important for the recovery process. The physiotherapist can measure objectively the end of a phase of recovery, how the patient performs at the end of the phase and how long it took to get there.

This is important in the recovery process as the patient does the same exercises as in the start of the phase and can compare the progress he's done in that phase.

For the physiotherapist it's a way of standardizing the outcomes of the recovery and measure individual milestones in the recovery process.

If the exercise is not done within the limits defined by the physiotherapist, the repetition is not counted and an audio, text and visual notification is sent to the patient to allow him to correctly perform the repetition for that particular exercise. This is done through a Patient exercise assistance module PEAD. This module is very important to the adherence and progress of the patient as it notifies the patient what he's doing wrong and when he is doing a wrong movement.

There are different types of assistance given to the patient performing the exercises.

The first type is the video assistance where a video of the exercise is shown, before the patient is expected to perform it. This is done to insure the patient remembers the exercise needed to be done.

The second type of visual assistance is the shadow of the movement: during the exercise a pre-generated shadow shows the patient how to perform the exercise. Having a standard shadow that would show how the joint of the patient should move would work only partially as each exercise is customized based on the progress of the recovery. For example, a patient immediately after a surgery can flex a joint only 50 degrees but after two weeks after the surgery he can flex for example to 90 degrees. This means the shadow has to be reconstructed based on the exercise constraints.

There is an algorithm built into the solution that allows a pre-recorded shadow of an exercise to be reconstructed when the constraints of that exercise are changed. The algorithm analyses the base shadow recording, segmenting it into key frames like the start of the movement, the peak of the movement and the return of the movement. This allows the reconstruction of the shadow for most of the exercises. For movements that are not linear the algorithm analyses two differently defined shadows (for example one with target constraints at 30 degrees and one with target constraints at 80 degrees) and generates the movement curves corresponding to the two segments above and below the joint. By comparing those movement curves, the algorithm interpolates the positions and regenerates the shadow based on the new constraints.

For exercises that are complex and have a sequence of key frames defined, the shadow is regenerated individually for each segment taking into consideration all the exercise constraints and the individual segments timing.

The third type of visual assistance is a 3D real-time joint representation on which the sensors are fixed. The 3D rendition is moving on the screen of the mobile device synchronized with the actual physical joint and shows in real-time how the joint angles change during the movement. This allows the patient to see the angles while he's doing the movement and get feedback on what he's doing. The difference from other solutions that use a 2D render is that all the angles of the joint are being rendered, not only one of them, like in the 2D representation. By having the relevant angles displayed on the mobile interface the patient has objective information about his/her momentary progress and can strive to improve.

The last types of notifications are the vibration notification, where the hardware sensors (HS) vibrate when a movement is performed outside the bounds set by the physiotherapist, and respectively audio notification where a generated voice is telling the patient what is wrong with the movement he's just performed.

On the Webserver, WS, a machine learning procedure and its related algorithm MLA is implemented for exercise program changes and program evaluation.

The progress of the patient's flexion is automatically analyzed against patterns already existing in the database. If the algorithm detects the patient has progressed faster or slower than the exercise program he has scheduled, it generates an alert for the physiotherapist notifying the situation and asking for a change of program. If the patient has progressed faster, the physiotherapist is asked if the patient can be scheduled to complete exercises more advanced. If the patient has progressed slower, then the physiotherapist is asked if the current program should be repeated and the patient be notified.

The machine learning algorithm MLA learns from the responses that the physiotherapist sends and allows the system to show more relevant notifications.

An end result of the machine learning algorithm is to increase the adherence of the patients to the exercise program and detect which exercises work best for a category of patients and offer better outcomes than others for any patient.

Another important use of the algorithm is detecting patterns in patient exercise schedule and profile the patient into exercising categories. This allows for better scheduling of the exercises and automatic personalization of the patient schedule. For example, if the patient is profiled as one that will have low adherence over the program, then some exercises can be switched to some that may be easier to perform.

Data gathered from the sensors consist of, but is not limited to, joint position and orientation, physical movement angles values (flexion angle, hip, elbow, shoulder, etc angle, abduction angle, hip, elbow, shoulder, etc. rotation angle), acceleration, speed, time spent between repetitions and per set of exercises, maximum angle values during a repetition, correctness grade, detected initial and final posture.

The Web server acts also as a physiotherapist interface WPTI to the cloud-based database which stores all the exercise/programs data and the patient data. It allows defining new exercises by setting movement constraints and changing existing exercises. Continuous synchronization with the physiotherapist and patient mobile applications ensures the exercise database is always up to date.

The web platform allows exercises and exercise programs to be defined by the physiotherapist or by the clinic. There are standard public programs defined and available for all the clinics and physiotherapists, and private programs that can be seen only by the clinic.

The web platform allows the physiotherapist to see all the exercises done by the patient, the progress of the recovery and adjust the program scheduled for the patient.

The progress of the recovery can be measured in the first weeks of the recovery by the flexion increase. This is the phase where the patient tries to recover the lost flexion due to the injury. In this phase all progress and exercises are targeted towards the recovery of function for the patient. The phase is assumed to be completed when the patient recovers flexion to the values previous to the injury.

The next phase of recovery is targeted towards strengthening the muscles and recovering to the previous state before the injury. The progress in this phase is measured by the number of exercises done, the increased duration of isometric exercises and the increased weights used for the training.

### CITATION LIST

### Patent Literature

1. US 2012/0190505 A1—Method and system for monitoring and feed-backing on execution of physical exercise routines;

2. WO/2007/125344—Exercise monitoring system and method;

3. US 20170143261—System and methods for monitoring physical therapy and rehabilitation of joints;

4. US20170136296A1—System and method for physical rehabilitation and motion training;

5. US20080262772A1—System and a method for motion tracking using a calibration unit;

6. EP3096685A1—System and method for mapping moving body parts

### Non Patent Literature

7. R. Takeda, G. Lisco, T. Fujisawa, L. Gastaldi, H. Tohyama, S. Tadano-Drift Removal for Improving the Accuracy of Gait Parameters Using Wearable Sensor Systems; https://www.ncbi.nlm.nih.gov/pmc/articles/PMC4299060/

1. A system for optimized physiotherapy monitoring of joints, built up within a mobile application and including the set of two motion sensing devices HS mounted above and below the injured joint of a patient, the mobile application MobApp that receives sensor data and implements the physiotherapists interface MPTI and the patient interface MPI within the Patient exercise assistance module PEAD, accessible using the corresponding login parameters, the Exercise definition module EDM, the assessment module ASD and the movement analysis module MAM, and respectively the web platform WS hosting the cloud-based database DB which keeps the physiotherapist, patient and exercise data, characterized by the fact that the mobile application includes the Calibration and compensation unit CCU, and the web server hosts the Machine learning unit MLU.

2. A system according to claim 1 where the Calibration and compensation unit CCU is arranged to allow for a free placement of the motion sensors above and below the affected joint based on the detection of the physical axis of the joint.

3. A system according to claim 1 where the Calibration and compensation unit CCU is arranged to dynamically minimize the linear gyro accumulation drift.

4. A system according to claim 1 where the Machine learning unit MLU is arranged to learn from the responses of the physiotherapist and the quality of the exercises done by the patient, thus allowing the system to optimise the exercises program and identify relevant patterns in exercise schedule, improving relevant notifications, and profiling the patient into exercising categories and automatic personalization of the patient schedule.

5. A method for an optimized physiotherapy monitoring of joints comprising:—processing data from the movement sensors via the calibration procedure, by minimizing the influence of the positions of the motion sensors on the body;—minimizing the influence of the dynamic drift of the gyroscope;—providing the movement analysis procedure;—providing the exercise definition procedure;—providing the patient exercise assistance procedure;—providing the machine learning procedure.

6. A method according to claim 5 where the calibration procedure implemented by the calibration algorithm CALIB, supposes processing the following steps:—determine the principal flexion axis;—calculate initial corrections from static pose;—correct the sensor data recorded during calibration with the above parameters;—run muscle artefact correction algorithm over the corrected data;—extract muscle artefact correction data;—determine algorithm initial error.

7. A method according to claim 5 where the drift compensation procedure implemented via the drift compensation algorithm DCA, has the following steps:—calculation of the double derivative of the angles returned by the gyroscope;—in order to reconstruct the angle, a double integration is used which adds two sets of constants, one set for each integration operation;—determination of the first set from the samples where the joint is not moving, in the static pose phase, where for those samples it's equal to zero;—determination of the second one as the initial orientation;—drift compensation when the readings of the two sensors are synchronized.

8. A method according to claim 5 where the movement analysis procedure implements the following steps:—analysis of the processed sensor data, calibration data and exercise definition data;—generation of anatomical, and dynamic sensors correction;—exercise detection;—assessing the quality movement by generating the assessment data and respectively the repetition and movement quality data.

9. A method according to claim 5 where the exercise definition procedure implements he following steps:—detection of initial posture;—detection of target posture;—detection of mainframes between initial and target posture;—extraction of continuous, angle and isometric constraints;—extraction of timing and exercise duration;—segmentation of complex exercises.

10. A method according to claim 5 where the patient exercise assistance procedure includes a visual assistance on the 3D real-time joint representation on which the sensors are fixed, and has the following steps:—rendering the 3D image joint representation on the screen of the mobile device synchronised with the actual physical joint;—showing in real time how the joint angles change during the movement;—allowing the patient to see the angles while he's doing the movement and getting feedback on that.

11. A method according to claim 5 where the machine learning procedure implemented via the machine learning algorithm MLA, which, based on the input data and outcomes data stored in the database DB, implies processing the following steps:—find best exercises in terms of adherence vs. exercises;—find patterns in quality vs. adherence exercises;—analyze physiotherapist notifications;—analyze pain data in relation to adherence;—analyze best outcomes exercises for a patient affection;—find best assessment exercises.

12. A computer program comprising instructions which, when the program is executed by a computer, cause the computer to carry out the method of claim 5.
